(19) United States
(12) Patent Application Publication
(10) Pub. No.: US 2006/0052805 A1
(43) Pub. Date: Mar. 9, 2006

(54) Tongue scraper and brush

(76) Inventor: James L. Cwik, Willowbrook, IL (US)

Correspondence Address: MICHAEL BEST & FRIEDRICH LLC, 401 NORTH MICHIGAN AVENUE, SUITE 1900, CHICAGO, IL, 60611-4212, US

(21) Appl. No.: 10/935,295

(22) Filed: Sep. 7, 2004

(51) Int. Cl. A61B 17/24

(52) U.S. Cl. 606/161

(57) ABSTRACT

A tongue scraper and brush device including a handle, a head, and a neck located therebetween, the head having a top member with opposite brush and scraper faces, a scraper rib upstanding from the scraper face, and a row of bristle tufts upstanding from the bristle face. Two or more rows of ribs and two or more rows of bristle tufts or elastomeric fingers may be used and a longitudinal opening may be provided between adjacent rows of ribs and tufts. The scraper rib may be scalloped.

### FIELD OF THE INVENTION

The present invention relates generally to devices used to help improve dental hygiene, and more particularly to a tongue scraper and brush that is particularly effective and easy to use in cleaning the tongue.

### BACKGROUND OF THE INVENTION

While in modern times the American Dental Association has recommended tongue cleaning for better oral hygiene, it is believed that tongue scraping for dental hygiene purposes was practiced in ancient Asian cultures and in the Roman Empire. Even today tongue scraping is more commonly practiced as part of a dental hygiene regimen in Asia and Europe than in the United States. This may be due to the lack, until now, of a tongue scraper that satisfies the requirements of many potential tongue scraper users in the United States and elsewhere.

Many people who generally practice good oral hygiene brush and floss their teeth on a consistent basis, neglect to clean their tongue. This is unfortunate for the reasons explained below. Furthermore, while everyone is advised to clean their tongue as part of their oral hygiene practices, heavy smokers, mouth breathers and people who use dentures are particularly advised to include tongue scraping in their dental hygiene regimen.

The tongue is covered with various types of papillae and taste buds scattered over the upper and side surfaces of the tongue. The posterior section of the tongue, which is located near the throat, is covered with muciparous glands and lymph follicles. These and other structures and surface characteristics of the tongue create a variety of furrows, grooves, folds, pits and other topological features that tend to trap food debris and bacteria. Some of the anaerobic bacteria in the tongue such as Fusobacterium and Actinomyces breed deep within the fibers of the tongue and excessive amounts of bacteria are associated with halitosis. More particularly, microbial metabolism generates sulfurous compounds from decaying oral bacteria, fungi, dead skin cells and food particles buried in the tongue's surface. Also, it is believed that the surface of the tongue is one of the main breeding grounds and reservoirs for bacteria that attack teeth and gums.

While natural tongue movement causes the anterior portion of the tongue to rub up against the hard palate producing a cleansing action that reduces bacterial accumulation, the posterior portion of the tongue only gently brushes against the palate and does not produce significant cleansing action. Thus, cleansing the surface of the tongue with a tongue scraper, particularly its posterior portion, as part of regular oral hygiene practices, will reduce the presence of the odor-causing agents and lower the bacterial count in the mouth. This will not only help control halitosis, it likely will have a positive effect on the health of gum tissues. Tongue scraping may also improve taste acuity by maintaining clear pathways to the taste buds. Finally, a toothpaste with antibacterial properties may be used in conjunction with the tongue scraping to help kill off many of the odor-causing bacteria and to neutralize malodorous volatile sulfur compounds produced by anaerobic bacteria.

It is therefore an object of the present invention to provide a tongue scraper and brush that is easy and comfortable to use.

It is a further object of the present invention to provide a tongue scraper and brush that is particularly effective in penetrating and removing debris and bacteria from the furrows, grooves, folds, pits and other topological features.

Yet another object of the present invention is to provide a tongue scraper and brush that produces effective cleansing of the tongue with minimum pressure, thereby decreasing the gagging reflex associated with many other tongue scrapers.

Still another object of the present invention is to provide a tongue scraper and brush that is easy to clean after each use.

A still further object of the present invention is to provide a tongue scraper and brush which facilitates the application of tongue cleansing compounds such as antibacterial dentifrice prior to tongue scraping.

### BRIEF SUMMARY OF THE INVENTION

The present invention comprises a tongue scraper and tongue device having a handle, a head and a neck located therebetween. The head has a top member oriented generally perpendicularly to the longitudinal axis of the handle with opposite brush and scraper faces. One and preferably two or more scraper ribs are upstanding from the scraper face of the top member. One and preferably two or more rows of bristle tufts are upstanding from the bristle face of the top member. An elongated lateral passage may be provided between pairs of rows of the bristle tufts and pairs of scraper ribs to facilitate cleaning the brush. Also, a shelf surface is provided at the proximal side of the base of each scraper, for collecting and trapping debris and bacteria as they are removed from the tongue surface.

The handle of the tongue scraper and brush is cigar-shaped to facilitate rotating preferably first the brush side of the device against the user's tongue and then the scraper side against the tongue. Also, thumb and finger rests are preferably provided on opposite sides of the handle corresponding respectively to the scraper side and bristle side of the top member.

### BRIEF DESCRIPTION OF THE DRAWINGS

For a more complete understanding of the present invention and further objects and advantages thereof, reference is made to the following description, taken in conjunction with the accompanying drawings, in which:

FIG. 1 is a perspective view of a tongue scraper and brush in accordance with the present invention;

FIG. 1A is a cross-sectioned view of the handle of the tongue scraper and brush of FIG. 1 taken above lines 1A-1A in FIG. 1;

FIG. 2 is a perspective view of the handle at the proximal end of the tongue scraper and brush of FIG. 1;

FIG. 3 is an elevation view of the brush side of the head of the tongue scraper and brush of FIG. 1; and

FIG. 4 is a perspective view of the scraper side of the head of the tongue scraper and brush of FIG. 1.

### DETAILED DESCRIPTION OF A PREFERRED EMBODIMENT OF THE INVENTION

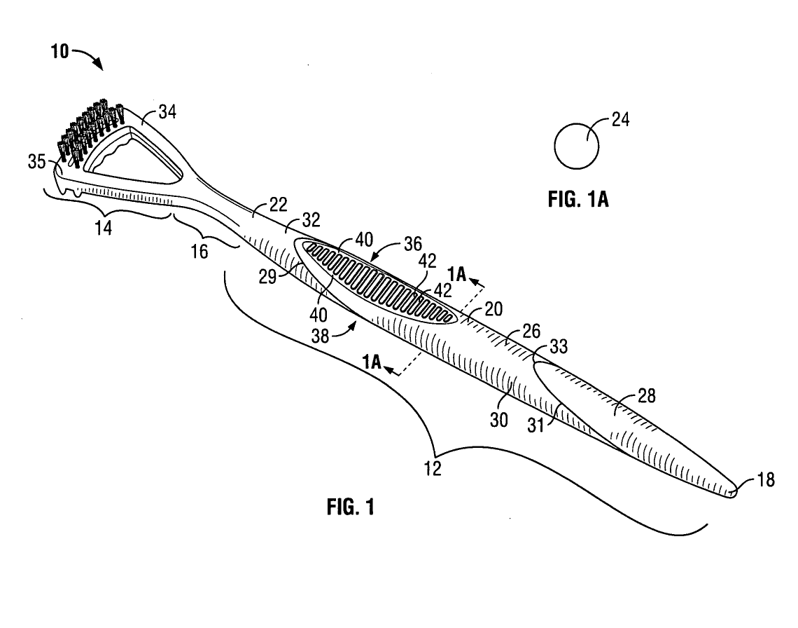

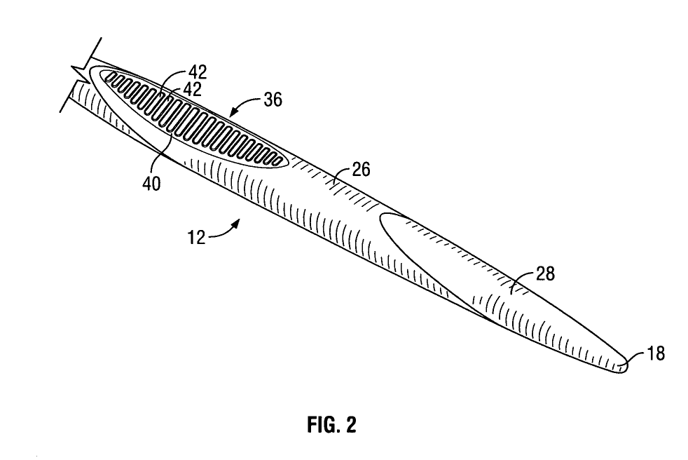

A tongue scraper and brush 10 in accordance with the present invention is illustrated in FIG. 1. The head may be angled about 5° to the longitudinal axis of the handle. This angle may be increased up to about 20° if desired. The device includes a handle 12 at its proximal end, a narrow tapered head 14 at its distal end to minimize the gagging reflex and a neck 16 located therebetween. Preferably, neck 16 will be flexible and resilient enough to bend if too much pressure is applied when the device is used and then to return to its original shape.

Handle 12 is cigar-shaped, beginning at its tapered proximal end 18, bulging outwardly towards the center portion 20 of the handle where the handle tapers again toward the distal end 22 of the handle. The handle should be long enough to enable the head of the device to reach the posterior third of the tongue. Handle 12 flows smoothly into the neck 16 of the device. Along its length, the handle has a circular cross-section as seen in section 24 of FIG. 1A, which is taken at the thickest portion of the handle (mid-point 20). Sections taken either proximally or distally of the handle middle will be similarly circular in profile, but of a smaller diameter due to the tapering of the handle toward its proximal and distal ends.

In the illustrated embodiment, handle 12 includes three handle sections, a proximal section 28, an intermediate section 30 and a distal section 32. These sections are made of contrasting colors so that they are well-defined. The sections meet along ellipses 29 and 31 that are oriented obliquely to the longitudinal axis of the handle. Ellipses 29 and 31 may be thought of as being formed by imaginary planes that intersect the handle surface along the ellipses. Proximal section 28 is positioned in the illustrated embodiment of the handle to have its greatest exposure parallel to the surface 35 of the brush side 34 of head 14. Additionally, the distal end 33 of ellipse 31 is oriented toward the brush side 34 of the head of the device.

Handle 12 also includes a pair of thumb and finger rests 36 and 38 on opposite sides of the handle. More particularly, thumb and finger rest 36 is on the brush side of the device whereas thumb and finger rest 38 is on the scraper side (FIG. 4) of the device. Both finger and thumb rests include an elastomeric inset 40 and a series of laterally-oriented elongated protruberances 42 which extend across the thumb and finger rests.

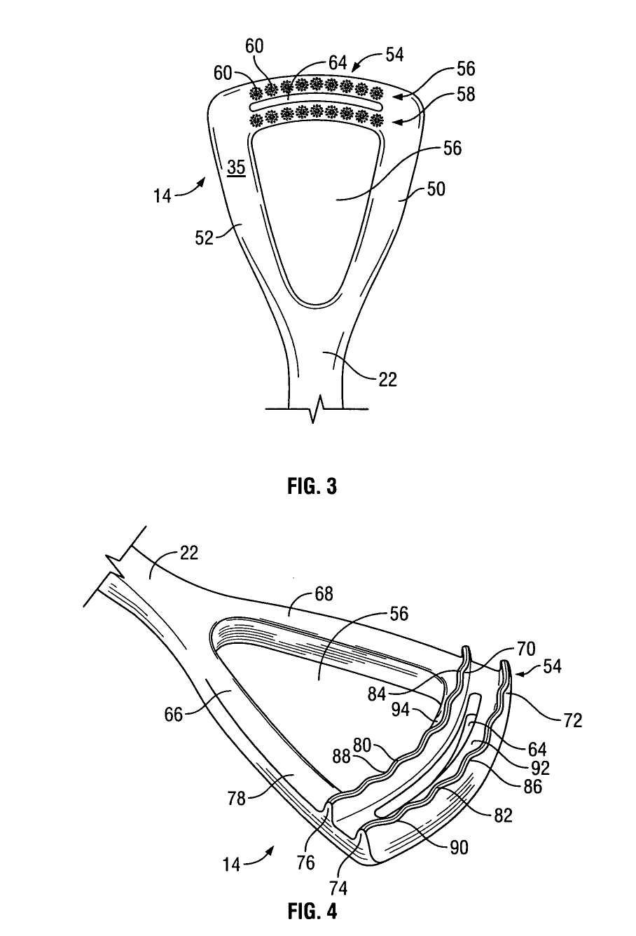

Turning now to the brush side 34 of head 14, it may be seen in FIGS. 1 and 3 that the head includes lateral sides 50 and 52 and a gently distally curved top member 54 generally perpendicular and symmetrically disposed with respect to the longitudinal axis of the handle. The lateral sides and top member define an enclosed triangular area 56 in the head. The corners of this triangular area are rounded, as shown, to minimize the presentation of sharp corners that might unnecessarily accumulate debris or present a danger of injury. This head profile facilitates reaching the back of the tongue.

Top member 54 includes rows 56 and 58 of upstanding bristle tufts 60 that follow the gentle outward curvature of top member 54. While two generally parallel rows of bristle tufts appear in the illustrated embodiment of the invention, and are preferred, one row or more than two rows can be used. Also, some or all of bristle tufts 60 may be replaced with elastomeric fingers. Such elastomeric fingers may be made from thermoplastic elastomers that have sufficient stiffness for tongue massage and cleaning but are sufficiently soft to provide comfort and avoid irritation during use. Suitable thermoplastic elastomers include polytheramides, polyesters, styrene-ethylene-butylene-styrene block copolymers, styrene-ethylene-propylene-styrene block copolymers, styrene-butadiene-styrene block copolymers, and styrene-isoprene-styrene block copolymers. In the illustrated embodiment an elongated lateral passage 64 is formed in the head between the two rows of bristles or fingers. This passage, which extends through to the opposite side of the head, functions as described below, to facilitate cleansing. When more than two rows of bristle tufts are provided, it is preferred that elongated lateral passages are provided between each successive pair of rows of bristle tufts.

In FIG. 4, sides 54, 66 and 68, as well as triangular opening 56 and elongated passage 64 are seen from the opposite, scraper side of the head. Unlike the bristle side of the head, however, on this side, two upstanding, gently outwardly curved scraper ribs 70 and 72 are shown, generally following the curvature of top member 54, juxtaposed on either side of passage 64. Note that a single scraper rib or more than two scraper ribs can be provided. Scrapers 70 and 72 are rounded along their top edge as shown and comprise upstanding ribs 74 and 76 that project generally perpendicularly from the surface 78 of the scraper side of the head. In the currently preferred embodiment of the invention, the scraper ribs extend about 3 mm from surface 78. These ribs may, however, be from about 0.5 mm to about 10 mm in height. Additionally, ribs 70 and 72 are scalloped to present wavy rounded edges 80 and 82 whose peaks 84 and 86 and valleys 88 and 90 help penetrate the nooks and crannies in the surface of the tongue to aid removal and retention of debris. The peaks and valleys of wavy rounded edges 80 and 82 may be offset if desired to enhance the lifting action. As the debris is removed it collects and is effectively trapped along shelf surfaces 92 and 94 at the proximal side of the base of each of the scrapers and in the scraper valleys. This prevents the collected debris and bacteria from being spread in the mouth. This scraper configuration effectively lifts off the debris and bacteria, but is not sharp enough to cause injury to the tongue surface or other discomfort.

The body of tongue scraper and brush 10 may be made of polypropylene, polyethylene, copolyester or ABS resin or any other biocompatible, durable material with an easy-to-clean non-porous surface. The body also may be made of stainless steel if desired. Bristle tufts 60 may be made of nylon, or any other desirable material. The bristles are held in place by conventional means, such as metal anchor wire or insert molding. While it is preferred that bristle tufts 60 extend about 5 mm from the surface 35 of the brush side of head 14, the bristle tufts may extend from about 2 to about 10 mm from the surface 35 of the brush side of head 14, although other lengths may be used. The stiffness of the bristles is chosen in conjunction with the lengths of the bristle tufts to produce a debris-loosening effect with minimal pressure and without harming the tongue surface. In the illustrated embodiment of the invention the bristle tufts are trimmed flat. They may, however, be trimmed to a height curved across the width of the head, in a sawtooth pattern, in alternating heights, or in other configuration if desired. Also, while the bristles are shown in a perpendicular orientation to surface 78, they may be angled toward the proximal end of handle 12 from about −15° to +15°.

The tongue scraper and brush of the present invention may be used as follows.

1. Ideally just prior to toothbrushing the user picks up the handle, and smoothly rotates it, until the thumb and forefinger come to rest in the desired location on thumb and finger rests 36 and 38 with the remaining fingers gripping the proximal end of the handle. A user can easily achieve the desired orientation, tactilely sensing the tapered proximal tip of the handle and the thumb and finger rests as well as their elastomeric surfaces and elongated protuberances. Additionally, the user is signaled to pick out the desired one of the brush and scraper sides of the head by looking either at the head or at the orientation of any one of handle sections 28, 30 and 32.

2. With the lower fingers curved around the proximal end of the handle and the thumb and forefinger on rests 36 and 38, the user brings the device towards their mouth and preferably places the brush side of the head against the tongue surface first to loosen debris. A tongue cleansing agent or even optionally a conventional dentifrice may be first applied to the brush or directly to the tongue surface to facilitate the cleansing of the tongue. The cleansing process proceeds—with or without the cleaning agent—by starting as far back as possible and making brush strokes using an outward motion toward the front of the mouth with enough pressure to cleanse and without irritating the tongue surface.

3. Since brushing the tongue alone may loosen and redistribute the microorganisms on the tongue instead of actually removing them, it is important to follow the brushing with scraping. Thus, the handle is rotated in the user's hand so the wavy rounded edges 80 and 82 of scraper ribs 74 and 76 next engage the tongue surface. As in the case of the brush, the scraping is best done starting as far back as possible and making strokes using an outward motion toward the front of the mouth with enough pressure to scrape and lift off undesirable debris by the action of the two scalloped scrapers. The debris collects in scraper valleys 88 and 90 and on shelf surfaces 92 and 94.

4. When the user has completed the process, using either the scraper alone, the toothbrush alone, or both the toothbrush and scraper in sequence as preferred, the user withdraws the head of the device from the mouth and rinses it by passing a stream of water against the brush and scraper sides of the head. The stream of water passes through the triangular opening 46 in the head as well through passage 64, carrying with it any debris accumulated on the toothbrush bristles, or collected in the valleys 88 and 90 and shelf surfaces 92 and 94.

5. In an alternate embodiment, the user may wish to use the brush and scraper to clean the tongue and then tilt the device slightly upward so that the bristles contact the roof of the mouth to remove debris from that area.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A tongue scraper and brush device comprising:
a handle, a head, and a neck located therebetween;
the head including a top member oriented generally perpendicularly to the longitudinal axis of the handle and having opposite brush and scraper faces;
at least one scraper rib upstanding from the scraper face; and
at least one row of bristle tufts upstanding from the bristle face of the top member.

2. The tongue scraper and brush of claim 1 in which the handle is cigar-shaped.

3. The tongue scraper and brush of claim 1 in which the handle includes a pair of thumb and finger rests on opposite sides of the handle.

4. The tongue scraper and brush of claim 3 in which one thumb and finger rest is on the brush side of the handle and one thumb and finger rest is on the scraper side of the handle.

5. The tongue scraper and brush of claim 3 in which at least one of the thumb and finger rests includes an elastomeric inset.

6. The tongue scraper and brush of claim 3 in which at least one of the thumb and finger rests includes laterally-oriented elongated protuberances.

7. The tongue scraper and brush of claim 1 in which the top member is distally curved.

8. The tongue scraper and brush of claim 1 in which there are at least two generally parallel rows of bristle tufts upstanding from the bristle face of the top member.

9. The tongue scraper and brush of claim 8 in which there is an elongated lateral passage between each successive pair of rows of generally parallel bristle tufts.

10. The tongue scraper and brush of claim 1 in which there are at least two generally parallel scraper ribs upstanding from the scraper face.

11. The tongue scraper and brush of claim 10 in which there is an elongated lateral passage between each pair of generally parallel scraper ribs upstanding from the scraper face.

12. The tongue scraper and brush of claim 1 in which the scraper ribs are scalloped.

13. The tongue scraper and brush of claim 10 in which the scraper ribs are scalloped to present wavy edges with peaks and valleys and the peaks and valleys of the scraper ribs are offset with respect to each other.

14. The tongue scraper and brush of claim 1 in which a shelf surface is provided at the proximal side of the base of the scraper rib.

15. The tongue scraper and brush of claim 1 in which the bristle tufts extend about 2 to 10 mm from the surface of the brush side of the head.

16. The tongue scraper and brush of claim 1 in which the bristle tufts extend about 5 mm from the surface of the brush side of the head.

17. A tongue scraper and brush device comprising:
a handle, a head, and a neck located therebetween;
the head including a top member oriented generally perpendicularly to the longitudinal axis of the handle and having opposite brush and scraper faces;
at least two generally parallel scraper ribs upstanding from the scraper face; and
at least two generally parallel rows of bristle tufts upstanding from the bristle face of the top member.

18. The tongue scraper and brush of claim 17 in which the handle includes a pair of thumb and finger rests on opposite sides of the handle.

19. The tongue scraper and brush of claim 17 in which there is an elongated lateral passage between each successive pair of rows of generally parallel bristle tufts and each successive pair of generally parallel scraper ribs.

20. The tongue scraper and brush of claim 17 in which the scraper ribs are scalloped, to present wavy edges with peaks and valleys.

21. The tongue scraper and brush of claim 17 in which a shelf surface is provided at the proximal side of the base of the scraper rib.

22. The tongue scraper and brush of claim 17 in which the bristle tufts extend about 2 to 10 mm from the surface of the brush side of the head.

23. The tongue scraper and brush of claim 17 in which the bristle tufts extend about 5 mm from the surface of the brush side of the head.

24. A tongue scraper and brush device comprising:
a handle including a pair of thumb and finger rests on opposite sides of the handle, a head, and a neck located therebetween;
the head including a top member oriented generally perpendicularly to the longitudinal axis of the handle and having opposite brush and scraper faces;
at least two generally parallel rows of bristle tufts upstanding from the bristle face of the top member, the scraper ribs being scalloped to present wavy edges with peaks and valleys, the scraper ribs each further having a shelf surface at its proximal side;
at least two generally parallel scraper ribs upstanding from the scraper face.

25. The tongue scraper of claim 1 in which some or all of the bristle tufts are replaced with elastomeric fingers.

26. The tongue scraper of claim 17 in which some or all of the bristle tufts are replaced with elastomeric fingers.
